(19) United States
(12) Patent Application Publication
(10) Pub. No.: US 2021/0196498 A1
(43) Pub. Date: Jul. 1, 2021

(54) DEVICE FOR REHABILITATION OF CHILDREN WITH CHILDHOOD CEREBRAL PALSY (CCP)

(71) Applicant: Aleksandr Grigorievich MYSLEVTSEV, g. Xabarovsk (RU)

(72) Inventor: Aleksandr Grigorievich MYSLEVTSEV, g. Xabarovsk (RU)

(21) Appl. No.: 17/057,704

(22) PCT Filed: May 20, 2019
(86) PCT No.: PCT/RU2019/000347

§ 371 Date: Nov. 23, 2020

(30) Foreign Application Priority Data
Jun. 18, 2018 (RU) 2018122341

(51) Int. Cl. A61F 5/01

(52) U.S. Cl. CPC A61F 5/0116; A61F 5/0109

(57) ABSTRACT

The device for rehabilitation of children with childhood cerebral palsy (CCP) comprises a sleeve/tube (1) made of an elastic material and cuffs made of Velcro strips (2). A frequently occurring problem during rehabilitation of children suffering from CCP is their wrong posture while sitting on the floor. The patients find this position is very comfortable since it provides good stability. However, this results in both wrong position of joints and wrong muscle function being developed, in particular body movement and balance are maintained by means of support on the inside on the foot with the knee being turned inwards. An inverted “knee inward” leg position becomes fixated. The feet become twisted with the big toes pointing outwards, which enhances undesirable changes in the function of the knee and hip joints. The above-mentioned factors reduce the effectiveness of rehabilitation carried out by traditional methods. The proposed device for rehabilitation of children with CCP makes it possible to rectify the aforementioned disadvantages without significantly restricting the patients' mobility. The device is fitted onto the feet of the patient and fixed at the ankles by the cuffs made of Velcro strips (2). The device fixed in this manner on the feet of the patient can stretch with movement. The effect of the device is observed already after a few first months of the use thereof.

The invention relates to medicine and applies specifically to fixating the hip joints in the correct position, training the leg muscles to function properly, and forming a step reflex in children with ICP when sitting and moving on bent knees.

To this day, the problem of preventing ICP in young children with perinatal encephalopathy during the first months of their lives remains practically unsolvable. It is known that children with severe encephalopathies develop a reflex pathology, which is manifested in an augmentation of pathological tonic reflexes. The latter prevent the formation of physiological righting reflexes and determine the formation of pathological muscle synergies, which create the basis for further development of the contractures of appendicular joints and lead to pathology of the functional antigravitation system (FAS), and specifically, one of its key structures—the vestibular system. First, a delay in the formation of the vestibular system is manifested, and later the statics and locomotion impairment are developed.

Several ICP prevention methods are known, such as massage, exercise therapy, pharmacotherapy, and physiotherapy. However, implementation of these methods not always produces a therapeutic effect. Some special suits are common, which create a load onto one muscle group, while unloading other groups.

The invention is intended for rehabilitation treatment of the patients suffering from movement disorders of cerebral origin, and is based on the use of a device for treating postural and motor activity impairments.

Similar purpose devices are known from the prior art.

A one-piece suit for treating the ICP patients and patients with the aftereffects of cerebrocranial injury is known, which comprises a support and righting bandages for the shoulder girdles, lumbar region, lower back, and lower extremities fabricated in accordance with certain anthropometric dimensions of the user (see Russian Federation Patent No. 2131232, IPC: A 61 F 5/02, publication date: Jun. 10, 1999).

The main disadvantages of this known invention are associated with the operational complexity, bulkiness of the product, considerable weight and the need for precise fitting in accordance with the anthropometric measurements of a particular user. The suit is of little use when treating young children.

A therapeutic suit for the infantile cerebral paralysis patients is known, which comprises tights, a long-sleeve shirt, a waistcoat, a waist belt, and gloves made of a tight body-fitting fabric with interknitted spiraling high-density straps made of elastic threads to enable stretching when the patient moves in the direction opposite to the action of pathological hypertonia on the skeletal muscles of the extremities (see RF Utility patent No. 67441, IPC: A 61 F 13/00, publication date: Oct. 27, 2007).

The main disadvantage of the known utility model is the need for precise fitting of the suit in accordance with the anthropological measurements of a particular user. Regular wear of the suit causes inconveniences associated with putting it on, taking it off, and actually wearing such suit, which requires considerable muscular efforts to stretch the straps made of elastic threads.

A method for preventing the infantile cerebral palsy with perinatal encephalopathy and a device for implementing thereof is known, which involves affecting a functional antigravitation system by preventing an augmentation of the labyrinthine and cervical symmetric and asymmetric tonic reflexes and formation of the righting reflexes. The device comprises a pelotte for the upper shoulder girdle and chest, a bandage for the lower back, bandages for the hips, and a device representing a hollow container made of an elastic airtight material. The wall of the container has an opening provided with a device for hermetic sealing of said container (see RF patent No. 2221537, IPC: A 61 N 1/00, A 61 N 33/00, publication date: Jan. 20, 2004).

The main disadvantages of the known invention are the forcing effect required to achieve the maximum abduction of the shoulder girdles posteriorly and fixating them in such position for up to 20-30 minutes, as well as the maximum transverse abduction of the child's hips using a special device, which causes a sensible discomfort and pain, and deprives the child of the ability to move for the entire duration of the procedure.

The objective of the proposed invention is to create a device, which would make it possible to more effectively eliminate pathological tonic reflexes in children with encephalopathy, followed by further development of the righting reflexes, statics and locomotion, as well as increase the efficiency and simplify the existing rehabilitation programs, such as Bobath therapy, Vojta therapy, exercise therapy, massage, pharmacotherapy, and physiotherapy.

The technical result achieved by implementing the proposed invention consists in manufacturing a device, which would allow holding the feet of the ICP patient in a physiologically correct position, while conducting the rehabilitation procedure.

This result is achieved by the fact that according to the invention, a device for rehabilitation of children suffering from infantile cerebral palsy (ICP) comprises a sleeve/tube made of an elastic material with the cuffs made of Velcro-strips and sewn along the edges on both sides. Said device is adapted for fitting on the child's feet and securing around the ankles.

The invention is illustrated by the following drawings:

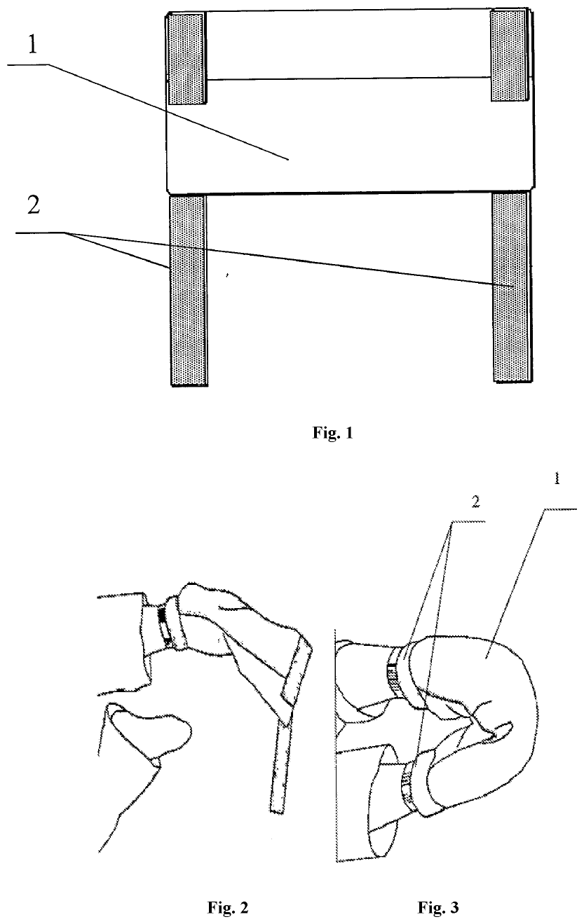

FIG. 1 shows a general view of the device for rehabilitation of children with infantile cerebral palsy;

FIG. 2 shows the device for rehabilitation of children with infantile cerebral palsy with the bandage on one foot;

FIG. 2 shows the device for rehabilitation of children with infantile cerebral palsy with the bandage in working position on both feet;

FIG. 4 shows a side view of the device for rehabilitation of children with infantile cerebral palsy, when the patient wearing a bandage is sitting in the “on the knees” position;

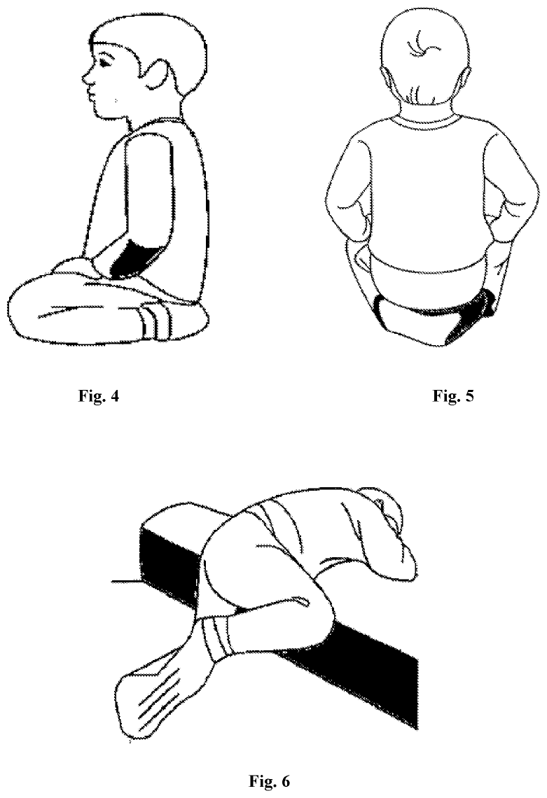

FIG. 5 shows a rear view of the device for rehabilitation of children with infantile cerebral palsy, when the patient wearing a bandage is sitting in the “on the knees” position;

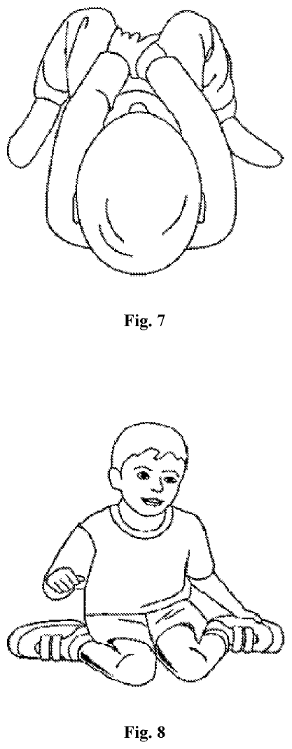

FIG. 6 shows the device for rehabilitation of children with infantile cerebral palsy, when the ICP patient wearing a bandage is in motion;

FIG. 7 shows a top view of the device for rehabilitation of children with infantile cerebral palsy, when the ICP patient is not wearing a bandage and is in a typical position;

FIG. 8 shows the device for rehabilitation of children with infantile cerebral palsy, when the ICP patient is not wearing a bandage and is sitting on the floor.

The device for rehabilitation of children with infantile cerebral palsy comprises a sleeve/tube (1) made of elastic material and cuffs made of Velcro strips (2).

The device for rehabilitation of children with infantile cerebral palsy operates as follows.

A common problem during rehabilitation of children with infantile cerebral palsy is their incorrect posture while sitting on the floor (see FIGS. 7 and 8). Such position is very convenient for the patients, since it provides good stability. This can be observed in most patients. At the same time, an incorrect position of the joints and improper functioning of the muscles are developed, since the movement and balancing of the body is performed by using the inner part of the foot as a support, while the knee is turned inward. An inverted “knee inward” position becomes fixated. The feet become turned out with the toes pointing outward, which enhances the negative changes in the function of the knee and hip joints. All these factors reduce the effectiveness of rehabilitation carried out by the most popular methods.

The proposed device for rehabilitation of children with ICP makes it possible to correct such problems without significantly limiting the patient's mobility. The device is fitted on the patient's feet and secured around the ankles with the help of the cuffs made of Velcro strips (2) (see FIGS. 2, 3, 4, 5, and 6). The device fitted on the patient's feet as described above has the ability to stretch during movement.

In this case, a positive effect is achieved, which includes:
establishing the correct position of the joints and functioning of the muscles; the feet are turned with the toes inward, the knees are turned outward, a supporting knee becomes the point of support during movement;
activating the alternating use of the legs when moving forward in the crawling position with the knees turned outward and foot toes turned inward;
training of the step muscles responsible for lifting the leg up;
simplifying further rehabilitation activities.

The use of the proposed configuration of the device for rehabilitation of children with infantile cerebral palsy (ICP) is effective, if the patient prefers a sitting position and continues to be in such position for a long time during the day. The device is fitted on the patient's feet for an hour daily during the first week. Later, the wearing duration is extended over the entire time of sitting. When changing positions, the device is removed from one of the feet, or removed completely. The effect from using the device is already observed after the first months of use.

The proposed invention can be used to train the leg muscles to function properly and to eliminate pathological tonic reflexes in children.

1. A device for rehabilitation of children with infantile cerebral palsy (ICP), characterized in that it includes a sleeve/tube made of an elastic material with the cuffs sewn along the edges on both sides and made of Velcro strips, while said device is adapted for fitting on the child's feet and securing around the ankles.
